(19) United States
(12) Patent Application Publication
(10) Pub. No.: US 2011/0259088 A1
(43) Pub. Date: Oct. 27, 2011

(54) HIGH WATER CONTENT FUEL DETECTION SYSTEM

(75) Inventors: Andrew FISHER, Cookeville, TN (US); Gregory D. SHOPE, Cookeville, TN (US); Mark T. WIECZOREK, Cookeville, TN (US); Chad M. THOMAS, Cookeville, MN (US)

(73) Assignee: Cummins Filtration IP, Inc., Minneapolis, MN (US)

(21) Appl. No.: 13/092,197

(22) Filed: Apr. 22, 2011

Related U.S. Application Data
(60) Provisional application No. 61/328,224, filed on Apr. 27, 2010.

(51) Int. Cl. G01N 33/18

(52) U.S. Cl. 73/61.43

(57) ABSTRACT

A water detection system in a fuel delivery system of an engine that helps to limit the occurrence of water related corrosion or damage to various engine components by detecting if a fuel supply has a high level of water content, and providing a notice that appropriate action should be taken. The system includes a fuel filtration module, a water sensor to sense high water content of a fuel supply, and a control device connected to the water sensor and receiving inputs therefrom relating to the water sensed by the sensor. The system also includes an indication source connected to the control device for communicating that a problem with the fuel exists if high water content is detected.

### PRIORITY DATA

This application claims the benefit of U.S. Provisional Patent Application Ser. No. 61/328,224 filed on Apr. 27, 2010, which is incorporated by reference in its entirety.

### FIELD

This disclosure pertains to a detection system within a fuel delivery system to determine if a fuel supply is contaminated with a high content of water. The disclosed system entails the use of a water sensor(s) located at one or more suitable locations in the fuel delivery system that is integrated with a suitable control device and a software routine to determine the water content of the fuel supply.

### BACKGROUND

High quantities of water in fuel is abnormal and indicates the fuel has been contaminated. In some cases, this contamination can inhibit the performance and life of, for example, a fuel filter, which will allow for unwanted water to flow downstream to the engine and associated equipment, for example fuel injection system equipment, causing corrosion and other associated damage.

In the case of a diesel engine, the vast majority of available fuel delivery systems incorporate a filtration system that utilizes an electrical sensor to determine a certain volume of water accumulated in the filter module's sump. The current technology only detects the level of water accumulated in the sump, regardless of the time in which it was accumulated. However, the rate of water accumulation can be a key indication of the quality of the incoming fuel.

### SUMMARY

A means is described for use in a fuel delivery system of an engine to limit the occurrence of water related corrosion or damage to various engine components by detecting if a fuel supply has a high level of water content, and providing a notice that appropriate action should be taken. The fuel delivery system can be any type of fuel delivery system used on any type of engine. Examples include, but are not limited to, a fuel delivery system of an engine used on a vehicle or on power generation equipment.

In one embodiment, a water detection system in an engine fuel delivery system includes a fuel filtration module, and a water sensor to sense water in the engine fuel delivery system. The water sensor senses level of water concentration in the fuel or the rate at which water is accumulating, for example in a sump of the fuel filtration module, and generates at least one water sensor output based thereon. A control device is connected to the water sensor and receives the at least one water sensor output. The control device is programmed with an algorithm to interpret the water sensor output with respect to water concentration or rate of water accumulation and generate at least one control device output signal. An indication source is connected to the control device and receives the at least one control device output signal for communicating, for example, that a problem with the fuel exists or conversely communicating that the fuel is not problematic.

The rate of water accumulation is a reflection of the water concentration in the fuel. So by sensing water accumulation rate, one can also measure a water concentration. Likewise, by sensing water concentration in the fuel, one can also measure a water accumulation rate. Therefore, the sensor can also be characterized as sensing both water concentration and water accumulation rate, and the control device programmed with an algorithm to determine both water concentration and water accumulation rate if one or the other is known.

The control device can be any device suitable for interpreting the water sensor inputs as described, including, but not limited to, an Engine Control Module (ECM), a controller, fluid management control module, or any suitable data/information processing device.

The water sensor is configured to sense either or both of water concentration in the fuel and the rate at which water is accumulating at some location in the fuel delivery system. More than one water sensor can be utilized. The water concentration can be sensed at any location(s) in the fuel delivery system one finds to be convenient.

Examples of suitable locations include, but are not limited to, a fuel inlet or fuel outlet of the fuel filtration module. The water accumulation rate can be sensed at any location(s) in the fuel delivery system where water that has been stripped from the fuel accumulates. Examples of suitable locations include, but are not limited to, a water sump of the fuel filtration module or in a water sump of a fuel tank. Various sensor technologies known in the art can be utilized.

### BRIEF DESCRIPTION OF THE DRAWINGS

FIG. 1 is a schematic diagram of one embodiment of the new system.

FIG. 2 is a schematic diagram of another embodiment of the new system.

FIG. 3 is a schematic diagram of another embodiment of the new system.

FIG. 4 is a schematic diagram of another embodiment of the new system.

FIG. 5 is a schematic diagram of another embodiment of the new system.

FIG. 6 is a flow chart showing an algorithm performed by a control device of the system.

### DETAILED DESCRIPTION

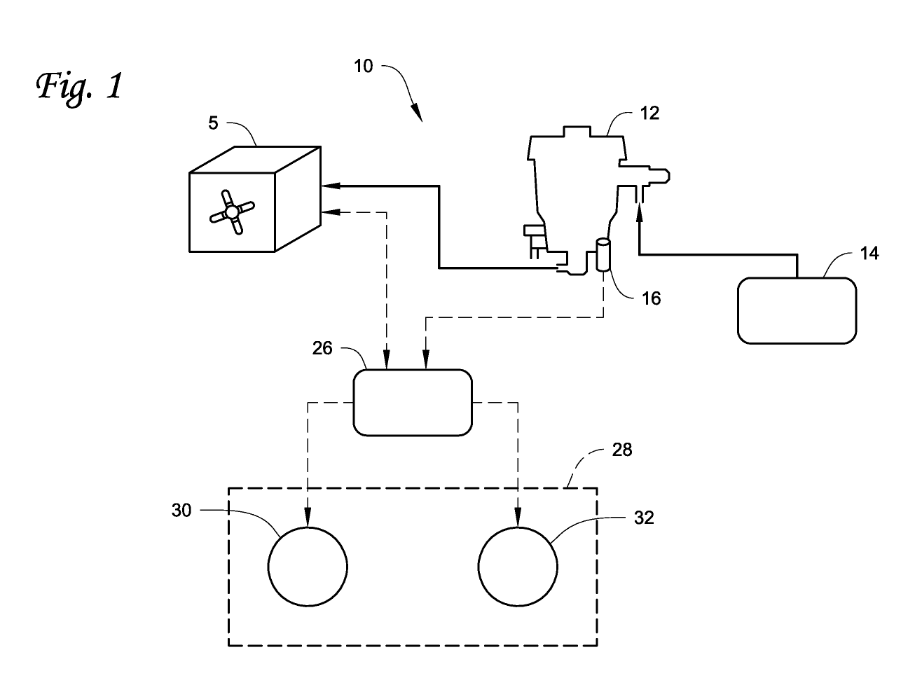

FIG. 1 illustrates a water detection system 10 as part of a fuel delivery system that delivers fuel to an engine 5. The system 10 is designed to determine the water content of fuel in the fuel delivery system prior to reaching the engine. The fuel delivery system can be any type of fuel delivery system used on any type of engine. Examples include, but are not limited to, a fuel delivery system of an engine used on a vehicle or on power generation equipment. One specific, non-limiting example of a fuel delivery system is a diesel fuel delivery system that delivers diesel fuel to a diesel engine of a vehicle.

The system 10 includes a fuel filtration module 12 that acts as a means to strip water and other particulates from the incoming fuel from a supply tank 14. In the illustrated embodiment, the filtration module 12 incorporates a sump, for example in a lower part thereof, for collection of water removed from the fuel, for example by the filter element in the filtration module. The sump requires draining when it becomes full. In fuel delivery systems that do not require water removal by the filtration module, the inclusion of a sump on the filtration module is optional.

The system 10 also includes a water sensor 16 that is suitably located in the fuel delivery system to sense water in the fuel. The water sensor 16 is designed to sense either or both of water concentration in the fuel and the rate at which water is accumulating at some location in the fuel delivery system. The water sensor 16 can be at any location(s) in the fuel delivery system one finds to be convenient. If the sensor 16 is to sense water accumulation rate, the sensor must be located in a sump area where water accumulates after being separated from the fuel.

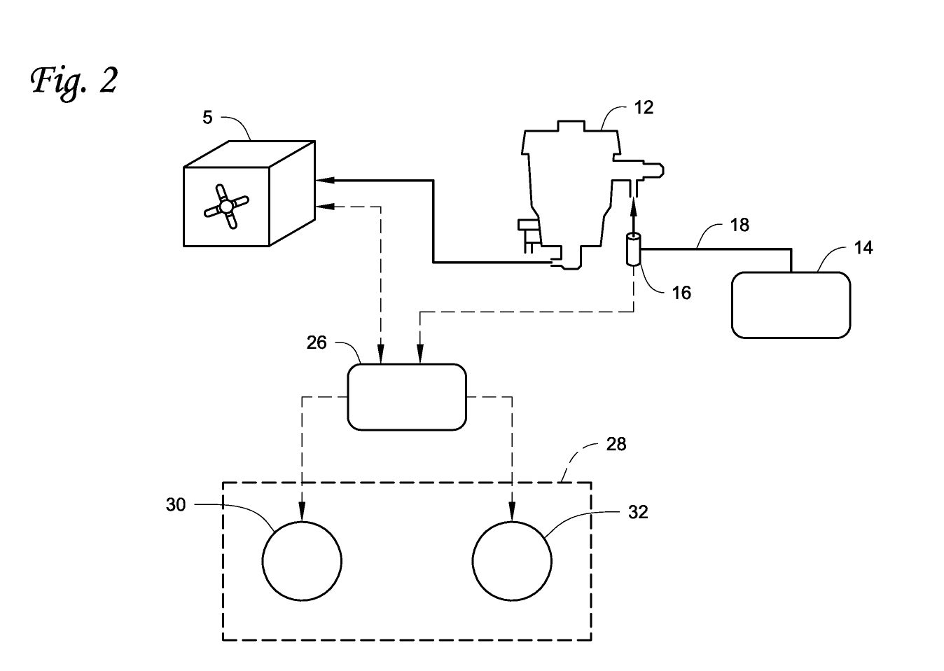

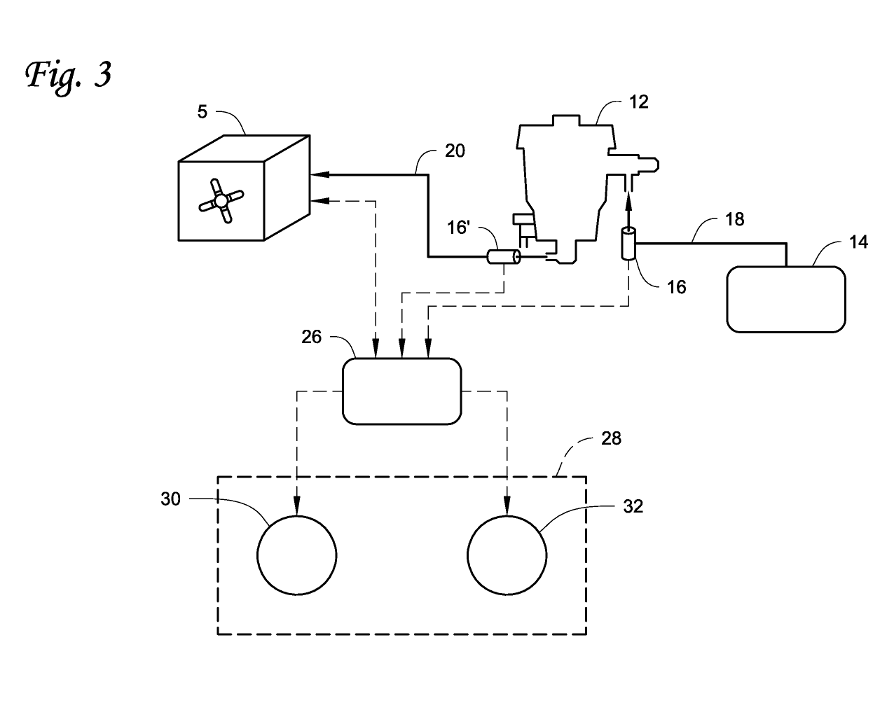

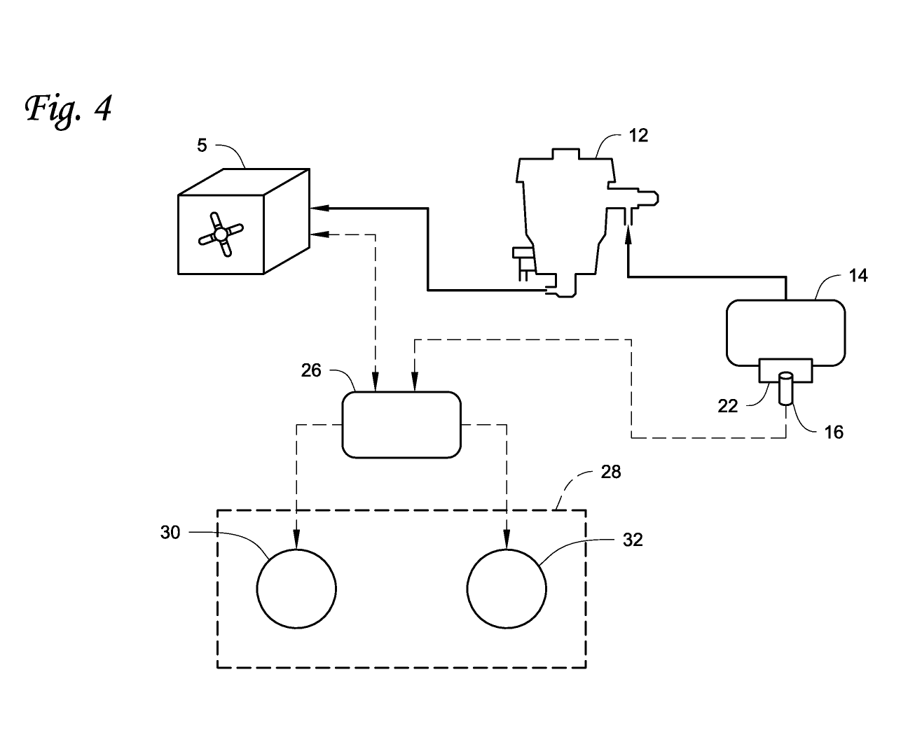

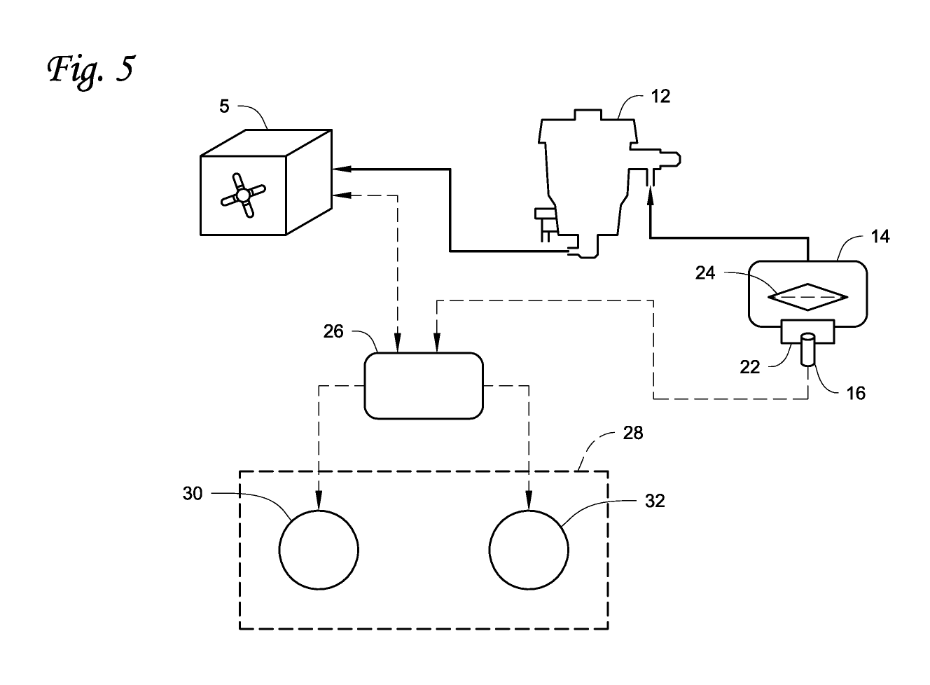

FIG. 1 illustrates the water sensor 16 positioned on the fuel filtration module 12 for sensing rate of water accumulation in the sump of the fuel filtration module 12. FIG. 2 illustrates an embodiment where the water sensor 16 is positioned to sense water concentration in the incoming fuel supply, for example in a fuel supply line 18 between the fuel tank 14 and the fuel filtration module. FIG. 3 illustrates an embodiment similar to FIG. 2, but with an additional water sensor 16′ positioned to sense water concentration in the outgoing fuel supply, for example in a fuel supply line 20 between the fuel filtration module and the engine 5. FIG. 4 illustrates an embodiment where the sensor 16 is located in a sump 22 of the fuel tank 14 for sensing the rate of water accumulation in the sump 22. FIG. 5 illustrates an embodiment that is similar to FIG. 4 but where the fuel tank 14 includes an intake fuel water separator 24. Other locations for the water sensor(s) are possible, and the various water sensor locations in FIGS. 1-5 can be used together in various combinations.

Various sensor technologies can be incorporated into the system 10 to sense water in the fuel. Suitable sensing technologies include, but are not limited to, in-line or sump capacitance sensing, float-style variable resistance sensing, light diffraction methods, light extinction methods, Fourier transform infrared spectroscopy, and other techniques generally known in the art. In addition, multi-level resistance sensing as described in copending application Ser. No. 61/328,391, titled MULTI-CONDUCTOR WATER IN FUEL SENSOR FOR FILL RATE DETECTION (attorney docket 20069.0185USP1), filed on Apr. 27, 2010, can be used.

The sensor(s) will translate the inlet fuel or sump mixture properties (dependent upon mounting scheme relative to the fuel delivery system) into subsequent output variations that are directly communicated to a suitable control device 26, for example an Engine Control Module (ECM), a controller, fluid management control module, or any suitable data/information processing device, for further processing.

The control device 26 is programmed with a specific algorithm to interpret the sensor inputs, thereby monitoring the water content of the fuel supply. The sensor readings will be processed to, for example, complete one of the following exemplary outputs:
1. Signal that draining of the fuel filtration module sump or fuel tank sump 22 is required
2. Alert the operator of high water content fuel (HWCF). In some embodiments, the poor quality fuel event may be succeeded by an alternate process, for example a process that can involve such outputs as engine de-rating and writing a permanent fault code to engine control memory. This event will require immediate service to protect the engine from any potential damage.

In addition, the control device 26 can also track the time and/or miles between notification being provided (such as to the driver in the case where the fuel delivery system is used on a vehicle) and when action such as draining the water from the fuel filtration module sump or the fuel tank sump is taken.

The system 10 also includes an indication source 28 that serves to communicate the control device 26 outputs to indicate a condition of the fuel. The indication source 28 can be, for example, one or more indicator lamps. The indication source 28 can, for example, inform the operator of either high water content fuel 30 based on the sensed water concentration or that the water accumulated within the filter sump or fuel tank sump requires draining 32. Appropriate action can then be taken to remedy the situation at hand. The indication source 28 can be, for example, mounted on a dashboard of a vehicle when the fuel system is used on a vehicle, or on a suitably located control panel for non-vehicle applications.

Although the figures illustrate two indicators lamps 30, 32, a single indicator lamp can be used to indicate a water concentration condition or a water accumulation condition. For example, the lamp could illuminate in one color to indicate a water concentration condition and illuminate in a different color to indicate a water accumulation condition. Alternatively, the lamp could illuminate continuously to indicate a water concentration condition and illuminate intermittently (i.e. blink) to indicate a water accumulation condition, or vice versa. The single lamp could use any means to distinguish the water concentration condition from the water accumulation condition.

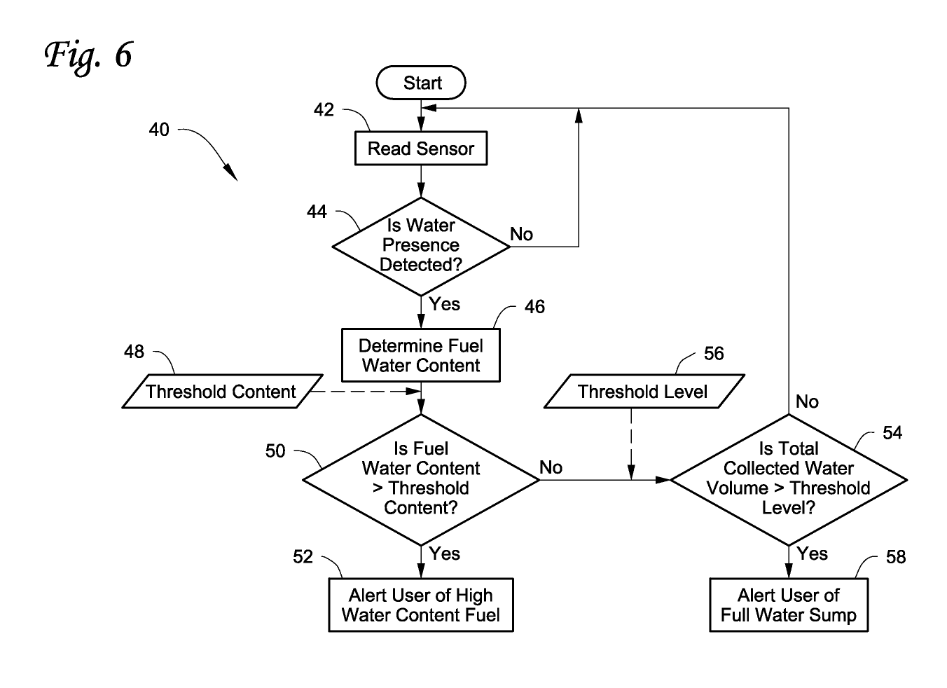

FIG. 6 illustrates an algorithm 40 run by the control device 26. The algorithm starts at 42 by reading the sensor 16. It is then determined 44 if water is present in the fuel. If the presence of water is not detected, the algorithm returns to the start. If water is detected, the algorithm proceeds to determine the fuel water content 46.

In the case of sensing water in the fuel supply lines 18, 20, the fuel water content can be determined by determining the water concentration, in parts per million, via an in-line capacitance sensing technique. In this technique, the sensor is an in-line capacitance sensor that outputs a signal that is directly correlated to the water content of the fuel contacting the sensor. The signal is then compared to a pre-defined threshold. When the threshold is exceeded, a timer is started to determine if the threshold is exceeded for a prescribed time limit. If the threshold is exceeded for the prescribed time limit, high water content fuel has been detected.

In the case of the filter module sump or the fuel tank sump, the fuel water content can be determined by determining the rate of water accumulation via a sump capacitance sensing technique. In this technique, the sensor is a capacitance style sensor that outputs a signal that is directly interrelated with the water content of the sump's volume mixture. The sensor output will be read at one point in time, and also at a later point in time. The accumulation rate is then determined by calculating the rate of change of the sensor outputs over the time between the measurements. The calculated rate is then compared against a pre-defined threshold or allowable rate of change. If the calculated rate of change is greater than the pre-defined threshold rate, high water content fuel has been detected.

In the case of float-style variable resistance sensing used in the sumps, the sensor employs a float whose position varies with the amount of accumulated water. The rate of water accumulation is calculated by determining the rate of change of the sensor outputs over the time between the measurements, and then comparing the rate against a pre-defined threshold or allowable rate of change.

In the case of multi-level resistance sensing, a resistance based level switch sensor is used to detect a rate of change of water accumulation in the filtration module sump or the fuel tank sump. The sensor is configured to have different water accumulation detection levels and change output signals at each level. The time interval between triggering of detection levels is compared against a pre-defined time interval, and if the detected time interval is greater than the pre-defined time interval, high water content fuel has been detected. Further information on the implementation of multi-level resistance sensing can be found in copending application Ser. No. 61/328,391, titled MULTI-CONDUCTOR WATER IN FUEL SENSOR FOR FILL RATE DETECTION (attorney docket 20069.0185USP1), filed on Apr. 27, 2010, which is incorporated herein by reference in its entirety.

Regardless of the technique used to determine the fuel water content, the fuel water content is compared to the threshold content 48, and if it is determined that the fuel water content exceeds the threshold content 50, the algorithm alerts the user of the high water fuel content 52.

If the result at step 50 is no, the algorithm proceeds to step 54 where the total collected water volume accumulated in the filtration module sump or the fuel tank sump, as measured by a conventional sensor that detects water level, is compared to a pre-defined threshold level 56. If yes, the algorithm alerts the user that the water sump is full 58 and needs to be drained. If no, the algorithm returns to the start.

The invention may be embodied in other forms without departing from the spirit or novel characteristics thereof. The embodiments disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A water detection system in a fuel delivery system of an engine, comprising:
a fuel filtration module;
a water sensor to sense water in the fuel delivery system, the water sensor sensing water concentration in fuel or rate at which water is accumulating and generating at least one water sensor output based thereon;
a control device connected to the water sensor and receiving the at least one water sensor output, the control device is programmed with an algorithm to interpret the water sensor output with respect to water concentration or rate of water accumulation and generate at least one control device output signal;
an indication source connected to the control device and receiving the at least one control device output signal.

2. The water detection system of claim 1, wherein the water sensor is positioned to sense water accumulation in a sump of the fuel filtration module or in a sump of a fuel tank of the fuel delivery system, or positioned to sense water concentration in a fuel inlet or fuel outlet of the fuel filtration module.

3. The water detection system of claim 1, wherein the engine is part of a vehicle or power generation equipment.

4. The water detection system of claim 1, wherein the water sensor senses water concentration in fuel.

5. The water detection system of claim 1, wherein the water sensor senses the rate at which water is accumulating in a sump of the fuel delivery system.

6. The water detection system of claim 1, wherein the fuel delivery system is a diesel fuel delivery system that delivers diesel fuel to a diesel engine of a vehicle.

7. The water detection system of claim 5, further comprising a water level sensor in the sump that detects water level in the sump, the control device is connected to the water level sensor and receives water level signals from the water level sensor, and the algorithm of the control device compares the received water level signals to a threshold level.

8. The water detection system of claim 1, wherein the indication source includes an indicator lamp that indicates high water content fuel or that water accumulated in a sump requires draining.

9. A method of detecting water in a fuel delivery system of an engine, comprising:
arranging a water sensor in the fuel delivery system to sense water concentration in fuel or rate at which water is accumulating in a sump of the fuel delivery system;
providing an output signal from the water sensor to a control device that is connected to the water sensor, the output signal being based on sensed water concentration or rate of water accumulation;
using the control device to interpret the water sensor output signal to generate at least one control device output signal; and
providing the at least one control device output signal to an indication source that is connected to the control device to provide an indication of the fuel water content of the fuel.

10. The method of claim 9, comprising arranging the water sensor to sense water accumulation in a sump of a fuel filtration module or in a sump of a fuel tank of the fuel delivery system, or arranging the water sensor to sense water concentration in a fuel inlet or fuel outlet of the fuel filtration module.

11. The method of claim 9, wherein the engine is part of a vehicle or power generation equipment.

12. The method of claim 9, comprising arranging the water sensor to sense water concentration in fuel.

13. The method of claim 9, comprising arranging the water sensor to sense the rate at which water is accumulating in the sump of the fuel delivery system.

14. The method of claim 9, wherein the fuel delivery system is a diesel fuel delivery system that delivers diesel fuel to a diesel engine of a vehicle.

15. The method of claim 13, further comprising arranging a water level sensor in the sump that detects water level in the sump, providing water level signals from the water level sensor to the control device, and using the control device to compare the water level signals to a threshold level.
